(19) United States
(12) Patent Application Publication
(10) Pub. No.: US 2025/0032164 A1
(43) Pub. Date: Jan. 30, 2025

(54) METHOD FOR OPTIMIZATION OF COOLING POWER FOR CRYOABLATION

(71) Applicant: Medtronic Cryocath LP, Toronto (CA)

(72) Inventors: Nicholas L. Nagel, Blaine, MN (US); Mark R. Pickard, Amery, WI (US)

(21) Appl. No.: 18/716,187

(22) PCT Filed: Nov. 24, 2022
(86) PCT No.: PCT/CA2022/051727

§ 371 Date: Jun. 4, 2024

Related U.S. Application Data
(60) Provisional application No. 63/287,679, filed on Dec. 9, 2021.

(51) Int. Cl. A61B 18/02; A61B 18/00

(52) U.S. Cl. CPC A61B 18/02; A61B 2018/00577; A61B 2018/00648; A61B 2018/00702; A61B 2018/00779; A61B 2018/00821

(57) ABSTRACT

Methods and systems for cryoablation cooling are disclosed. According to one aspect, a cryoablation cooling system includes cooling equipment having at least one operating parameter. The cryoablation cooling system also includes at least one external environment sensor, each of the at least one external environment sensors being configured to measure an environmental parameter external to the cooling equipment. At least one equipment sensor is included to measure an operating parameter of the cooling equipment. The cryoablation cooling system further includes a sub-cooler control system configured to determine an adjustment to at least one operating parameter of the cooling equipment to obtain a target cooling power based at least in part on the at least one measured external environmental parameter and the at least one measured operating parameter.

### CROSS-REFERENCE TO RELATED APPLICATION

The present application is related to and claims benefit under 35 U.S.C. § 119(e) from U.S. Provisional Patent Application Ser. No. 63/287,679, filed Dec. 9, 2021, entitled “METHOD FOR OPTIMIZATION OF COOLING POWER FOR CRYOABLATION” the entire contents of which being incorporated herein by reference.

### FIELD

The present technology is generally related to optimization of cooling power for cryoablation.

### BACKGROUND

Methods of thermally treating tissue are frequently used for a wide variety of medical purposes. For example, cryoablation, by which tissue may be destroyed, may be used to treat cardiac arrhythmia, to ablate tumors in the treatment of cancer, and for dermatological and obstetric procedures. Further, cooling tissue to sub-lethal temperatures is commonly used in electrophysiology studies.

Regardless of the tissue being treated, lesion permanency created by a cryoablation procedure may be an objective. In such cases, the target tissue is to be completely and permanently affected, which prevents the recurrence of the condition being treated. In the treatment of certain forms of arrhythmia, including atrial fibrillation (AF), permanent electrical conduction blocks are created at specific locations in the heart. Therefore, continuous, transmural lesions must be created, ensuring that all myocardial cells in the target tissue are destroyed. Specifically, myocytes are killed acutely by cold-induced injury through several mechanisms that can include cell membrane rupture due to ice formation, osmotic imbalance, dehydration, damage to the mitochondria, and delayed apoptotic processes.

The type and severity of the damage to tissue cells is influenced by several parameters of the treatment process. In a cryoablation procedure, for example, these parameters may include duration of the freeze, treatment temperature, cooling and thawing rate, and the number of freeze-thaw-freeze cycles. For example, extending the thawing phase of a cryoablation procedure by creating a temperature plateau at a mildly cold temperature, between approximately −20° C. and approximately −25° C., may result in more complete cell destruction and, therefore, a reduced likelihood of reconduction. Additionally, maintaining the treatment element in the target temperature range may reduce the occurrence of collateral damage by preventing the freeze zone from penetrating too deeply within the tissue.

Some current cardiac cryoablation systems operate at a controlled rate of refrigerant flow to the treatment device, resulting in an operating temperature that is the lowest achievable in the given conditions. In the current design of some cryoablation consoles, which provide and control cooling to a cryoablation catheter tip, effective cryotherapy is controlled using targeted flowrates. For example, a cryoablation console delivers pressurized refrigerant (such as N2O) with a controlled mass flow rate to the catheter. Heat is transferred from the surrounding of the catheter tip as the pressurized refrigerant expands and evaporates in the catheter tip. Cardiac cell lesions are created by the consequent rapid removal of heat. The cryoablation console then extracts the vapor refrigerant from the catheter and removes it through the facility's scavenging system.

A sub-cooler of the cryoablation console cools down the nitrous oxide by heat transfer before the coolant is injected into the catheter.

A problem with current technology is the variation of coolant temperature with the temperature of the environment surrounding the cryoablation system. In a cold environment, the charge pressure of the sub-cooler may fall below a charge pressure specified for the sub-cooler, and vice-versa, in a warmer environment the charge pressure of the sub-cooler may rise above the specified charge pressure for the sub-cooler. Due to this variability in charge pressure and the thermal load of the environment on the cryoconsole, the cooling power is affected.

### SUMMARY

The techniques of this disclosure generally relate to optimization of cooling power for cryoablation.

According to one aspect, a cryoablation cooling system includes: cooling equipment having at least one operating parameter; at least one external environment sensor, each of the at least one external environment sensors being configured to measure an environmental parameter external to the cooling equipment; at least one equipment sensor, each of the at least one equipment sensor being configured to measure an operating parameter of the cooling equipment; and a sub-cooler control system configured to determine an adjustment to at least one operating parameter of the cooling equipment to obtain a target refrigerant parameter based at least in part on the at least one measured external environmental parameter and the at least one measured operating parameter.

According to this aspect, in some embodiments, the adjustment is based on a transfer function that relates a given set of operating parameters and external environment parameters to a target refrigerant parameter. In some embodiments, the transfer function is predetermined based on measurements of the target refrigerant parameter for a number of sets of operating parameters. In some embodiments, determining at least one operating parameter based on the transfer function is further based on a difference between the target refrigerant parameter and a measured refrigerant parameter. In some embodiments, determining the adjustment includes: setting the target refrigerant parameter; and determining at least one operating parameter setting based on the transfer function and the set target refrigerant parameter. In some embodiments, the at least one measured operating parameter is one of a condenser fan speed, a compressor speed and a charging pressure. In some embodiments, the sub-cooler control system includes a sub-cooler proportional-integral-derivative (PID) controller configured to determine a control signal based at least in part on the target refrigerant parameter and a measured refrigerant parameter, the control signal controlling an operational parameter of the cooling equipment. In some embodiments, the target cooling power is determined based at least in part on a transfer function having inputs that include at least one measured operating parameter and at least one measured external environmental parameter. In some embodiments, the refrigerant parameter is one of a refrigerant pressure, a refrigerant temperature, and a refrigerant cooling power. In some embodiments, the at least one measured operating parameter is a pressure measured by a transducer and the at least one measured external environmental parameter is a temperature measured by a thermocouple. In the alternative to measuring the pressure by a transducer, the pressure may be set at a time of manufacture of the cryoablation cooling system.

According to another aspect, a method in a cryoablation cooling system includes: providing cooling equipment having at least one operating parameter; measuring via at least one external environment sensor an environmental parameter external to the cooling equipment; measuring via at least on equipment sensor an operating parameter of the cooling equipment; and determining an adjustment to at least one operating parameter of the cooling equipment to obtain a target refrigerant parameter based at least in part on the at least one measured external environmental parameter and the at least one measured operating parameter.

According to this aspect, in some embodiments, the adjustment is based on a transfer function that relates a given set of operating parameters and external environment parameters to a target refrigerant parameter. In some embodiments, the transfer function is predetermined based on measurements of the target refrigerant parameter for a number of sets of operating parameters. In some embodiments, determining at least one operating parameter based on the transfer function is further based on a difference between the target refrigerant parameter and a measured refrigerant parameter. In some embodiments, determining the adjustment includes: setting the target refrigerant parameter; and determining at least one operating parameter setting based on the transfer function and the set target refrigerant parameter. In some embodiments, the at least one measured operating parameter is one of a condenser fan speed, a compressor speed and a charging pressure. In some embodiments, the sub-cooler control system includes a sub-cooler proportional-integral-derivative (PID) controller configured to determine a control signal based at least in part on the target refrigerant parameter and a measured refrigerant parameter, the control signal controlling an operational parameter of the cooling equipment. In some embodiments, the target cooling power is determined based at least in part on a transfer function having inputs that include at least one measured operating parameter and at least one measured external environmental parameter. In some embodiments, the refrigerant parameter is one of a refrigerant pressure, a refrigerant temperature, and a refrigerant cooling power. In some embodiments, the at least one measured operating parameter is a pressure measured by a transducer and the at least one measured external environmental parameter is a temperature measured by a thermocouple.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a diagram of a cryoablation system including a cryoablation cooling system;

FIG. 2 is block diagram of an example control loop for controlling a cooling power delivered to a cryoablation catheter; and

FIG. 3 is a flowchart of an example process for controlling a cooling power delivered to a cryoablation catheter.

### DETAILED DESCRIPTION

Some embodiments advantageously provide methods and systems for providing greater control over the temperature of a treatment element of a catheter for cryoablation, enabling an operator to have greater control over a cryoablation procedure. In one embodiment, a system for compensating for environmental temperature changes in a cryoablation catheter is provided.

As used herein, the term “coolant” refers to the coolant of the sub-cooler and “coolant charge pressure” refers to the charge pressure of the coolant of the sub-cooler. In contrast, the term “refrigerant” refers to the refrigerant delivered from a source tank to the cryoablation catheter, “refrigerant pressure” refers to the pressure of the refrigerant received by the cryoablation catheter, and “refrigerant temperature” refers to the temperature of the refrigerant received by the cryoablation catheter. The term “refrigerant parameter” refers to the parameter of the refrigerant to be controlled, such as temperature, pressure, or cooling power.

In some embodiments, a target refrigerant flow rate is controlled via a first proportional integral-derivative (PID) controller. Also, a sub-cooler control system includes a sub-cooler controller, which may include a second PID controller, configured to control a temperature and/or pressure of the refrigerant delivered to the catheter by, for example adjusting a charging pressure of the coolant, fan speed of the sub-cooler condenser that condenses the coolant and/or a speed of a compressor that compresses the coolant. In some embodiments, the control of the refrigerant temperature and/or pressure is responsive to changes in temperature of an environment surrounding a cryoablation console housing the sub-cooler control system. By changing equipment operating parameters, such as a sub-cooler charging pressure, condenser fan speed or compressor speed, in response to a change in environmental temperature, a difference between a desired temperature and/or pressure of the refrigerant and the actual temperature and/or pressure of the refrigerant can be maintained below a specified upper variance limit over time or be constantly driven toward zero to maintain the desired temperature/pressure/cooling power of the refrigerant, notwithstanding the change in environmental temperature. Maintaining the desired refrigerant parameter (temperature/pressure/cooling power), enables more accuracy in achieving the intended treatment.

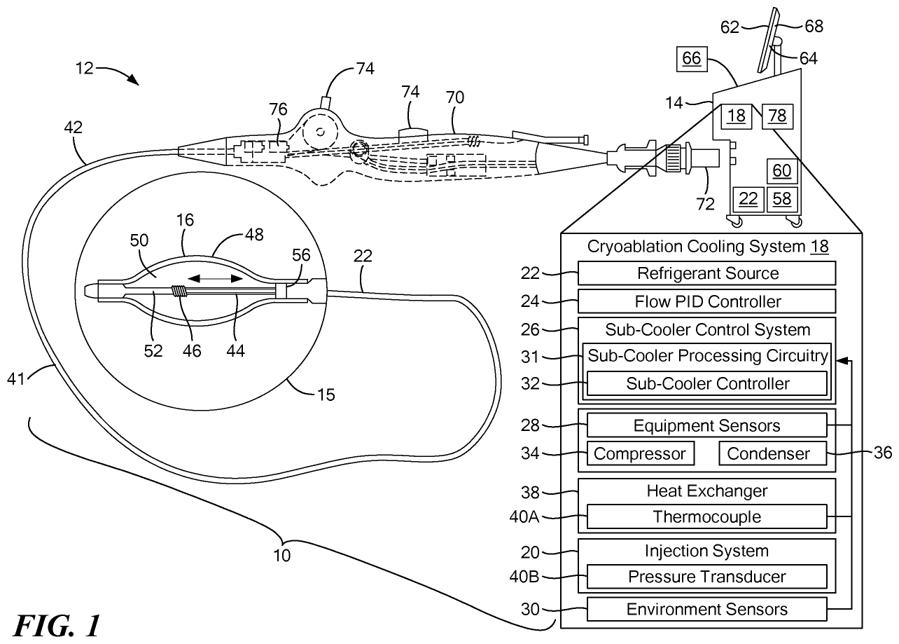

Referring now to the drawing figures in which like reference designations refer to like elements, FIG. 1 shows an example embodiment of a cryoablation system 10 including a catheter 12 accordance with principles disclosed herein. The system 10 also includes a cryoablation console 14 configured to provide refrigerant to the catheter 12. Refrigerant provided to the catheter 12 is delivered to a distal portion 15 of a treatment element 16 of the catheter 12. Further, the distal portion 15 of the catheter 12 may include one or more electrodes with mapping and/or ablation functionality (not shown).

The cryoablation console 14 includes a cryoablation cooling system 18 configured to regulate temperature and/or pressure of the refrigerant injected by an injection system 20 into the catheter 12 from a refrigerant source 22. The refrigerant source 22 may be a pressurized bottle of N2O, for example. A flow PID controller 24 controls a flow rate of the coolant from the refrigerant source 22 to maintain a constant rate of refrigerant flow (refrigerant mass transfer per unit time, for example) of the refrigerant to the catheter 12. The temperature and/or pressure and/or cooling power of the refrigerant injected into the catheter 12 by the injection system 20 may be regulated by a sub-cooler control system 26 that is configured to receive signals including signals from equipment sensors 28 and environment sensors 30. The signals received from equipment sensors 28 are representative of operating parameters of the equipment measured by the sensors 28. Similarly, signals received from environment sensors 30 are representative of environment parameters of an environment surrounding the measured by the sensors 28. Regulating the refrigerant parameter(s) based at least in part on environmental parameters provides greater stability and control of the refrigerant parameters, since the regulating as described below prevents deviation from a desired refrigerant parameter because of room temperature changes, for example.

The sub-cooler control system 26 may include a sub-cooler processing circuitry 31 which is configured to implement a sub-cooler controller 32. In operation, the sub-cooler control system 26 is adapted to determine an operating parameter of cooling equipment such as coolant charging pressure, speed of a compressor 34 and/or fan speed of a condenser 36, to achieve a constant mean value of the refrigerant parameter of the refrigerant injected into the catheter 12, with a variance that is less than a specified upper variance limit. As used herein the term “cooling power” may include a cooling power determined from a pressure and/or a temperature. Also, determining a pressure and/or temperature of the refrigerant injected, or to be injected, into the catheter 12 may be referred to as determining the cooling power. By adjusting the operating parameters of the cooling equipment, such as the charge pressure of the compressor 34 and/or fan speed of the condenser 36, the refrigerant parameter(s) of the refrigerant may be controlled.

The operating parameters may be determined according to the equipment sensors 28. For example, the charging pressure, speed of a compressor motor or a condenser fan motor may be sensed by electrical circuitry and/or components that sense an electrical current of the motor. Components and circuitry for sensing motor speed and charging pressure are known in the art.

In addition, there may be other equipment items not shown in FIG. 1 that have operating parameters that are sensed by the equipment sensors 28. Also, note that some environment sensors 30 may be external to the cryoablation cooling system 19. For example, the condenser may be located so that a working room temperature may be determined by a thermocouple device (one of the environment sensors 30) placed near the condenser fan, but external to the cryoablation console 14. Similarly, one of the environment sensors 30 may be a thermocouple placed near the compressor, for example, to sense a console working temperature. Advantages to having multiple environment sensors and multiple equipment sensors may include increased accuracy of control of the refrigerant pressure/temperature at the expense of greater computational complexity of the processing circuitry 31.

Thus, in addition to the equipment sensors 28, which sense operating parameters of cooling equipment of the cryoablation cooling system 18, which may include coolant charging pressure, compressor speed and/or condenser fan speed, environment sensors 30 sense environment parameters which may include temperature internal to the cryoablation cooling system 18 and/or external to the cryoablation console 14. As noted above, regulating the refrigerant parameters in response to environmental changes enables maintenance of a desired target refrigerant parameter in spite of the environmental changes.

The sensors 28 generate signals representative of the sensed operating parameters that are communicated to the sub-cooler control system 26. The sensors 30 generate signals representative of the sensed environment parameters, such as internal and external temperatures. These sensor signals are communicated to the sub-cooler controller 32 of the sub-cooler control system 26.

The sub-cooler controller 32 may continuously determine an error signal that may be indicative of a difference between a refrigerant parameter of refrigerant injected into the catheter 12 and a desired refrigerant parameter. The sub-cooler controller 32 may apply an adjustment to affect an operating parameter to drive the error signal toward zero and/or maintain the error signal to within a range about zero. In this way, the mean output response (refrigerant parameter) may be kept at a desired level and the variance about the mean may be kept below the upper variance limit. This provides advantages that include greater control over an extent of ablation (location, area and depth, for example) that may be administered by the operator of the system 10.

The heat exchanger 38 transfers heat between the coolant under control of the sub-cooler control system 26 and the refrigerant. A thermocouple 40A and/or pressure transducer 40B may determine a temperature and/or pressure of the refrigerant to be injected by the injection system 20 to the catheter 12. Although the thermocouple 40A and/or pressure transducer 40B are shown located at the heat exchanger 38 and at the injection system 20, they may be located elsewhere, in some embodiments. For example, a thermocouple 40A and/or pressure transducer 40B may be located within the catheter 12 and in electrical communication with the sub-cooler control system 26. Advantages of placing a thermocouple 40A at the heat exchanger 38 may include accessibility of the thermocouple 40A and ease of manufacture, whereas advantages of placing the thermocouple 40A within the catheter 12 may include a more accurate determination of the temperature of coolant delivered to the distal portion 15 of the catheter 12.

The thermocouple 40A and/or the pressure transducer 40B send a signal representative of the sensed temperature or pressure of the refrigerant to the sub-cooler control system 26.

The catheter 12 may include an elongate body 41 passable through a patient's vasculature and/or proximate to a tissue region for diagnosis or treatment. The elongate body 41 may define a proximal portion 42 and the distal portion 15, and may further include one or more lumens disposed within the elongate body 41 thereby providing mechanical, electrical, and/or fluid communication between the proximal portion 42 of the elongate body 41 and the distal portion 15 of the elongate body 41. For example, the medical device 12 may further include a refrigerant delivery conduit 44 traversing at least a portion of the elongate body 41 and towards the distal portion 15. The refrigerant delivery conduit 44 may be coupled to or otherwise extend from the distal portion 15 of the elongate body 41 into the treatment element 16. Although the treatment element 16 is shown in FIG. 1 as a balloon 48, it will be understood that the treatment element 16 may be any treatment element through which refrigerant may flow and that is capable of thermally affecting tissue.

One or more fluid injection elements 46 in fluid communication with the refrigerant delivery conduit 44 may be disposed within the interior chamber 50 of the balloon 48. A refrigerant injection element 46 may be of any configuration suitable for delivering refrigerant from the refrigerant delivery conduit 44 into the balloon 48. As a non-limiting example, a refrigerant injection element 46 may include a plurality of windings about a shaft or actuation element 52 within the chamber 50 of the balloon 48. Further, the refrigerant injection element 46 may include one or more openings or ports therein to provide for the delivery and, optionally, directed ejection of refrigerant from the refrigerant delivery conduit 44 to the chamber 50 of the balloon 48.

The refrigerant source 22, which may include one or more pressurized tanks which store the refrigerant under pressure, is in fluid communication with the refrigerant delivery conduit 44 and refrigerant injection element 46. The system 10 may also include an exhaust or scavenging system for recovering or venting expended refrigerant for re-use or disposal. The scavenging system may include a refrigerant recovery conduit 56 defining a passage for the recovery of expanded refrigerant, a refrigerant recovery reservoir 58, and a vacuum pump 60 for creating a pressure gradient to draw expanded refrigerant from the balloon chamber 50 into the refrigerant recovery conduit 56 and then into the refrigerant recovery reservoir 58 (that is, from the distal portion 15 of the catheter to the proximal portion 42 of the catheter 12 and into the cryoablation console 14). The system's refrigerant flow path may include at least the refrigerant delivery conduit 44 and the refrigerant recovery conduit 56, in addition to various other conduits and/or secondary flow paths. Further, although the refrigerant source 22 and the refrigerant recovery reservoir 58 each may each be located within or external to cryoablation console 14, they are referred to as being part of the cryoablation console 14 for simplicity. The cryoablation console 14 may also include pumps, valves, controllers or the like to recover and/or re-circulate fluid delivered to the fluid pathways of the catheter 12.

The cryoablation console 14 may include one or more controllers, processors, and/or software modules containing instructions or algorithms to provide for the automated operation and performance of the features, sequences, or procedures described herein. For example, the cryoablation console 14 may include one or more computers 62 that include one or more processors 64 for receiving signals from one or more sensors throughout the system 10, and or for the automatic, semi-automatic, and/or manual operation of the system 10. The one or more computers 62 may include one or more user input devices 66 by which a user can program system parameters such as the inflation and deflation of the balloon 48, circulation of refrigerant through the refrigerant delivery conduit 44 and refrigerant recovery conduit 56, and/or the operation of one or more electrodes or other thermal delivery elements.

The computer 62 may include a central processing unit, and memory. For example the computer 62 may comprise integrated circuitry for processing and/or control, e.g., one or more processors 64 and/or processor cores and/or FPGAs (Field Programmable Gate Array) and/or ASICs (Application Specific Integrated Circuitry) adapted to execute instructions. The processor 64 may be configured to access (e.g., write to and/or read from) the memory, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

Software executed by the processor 64 may be stored in the memory of the computer 62 or stored in external memory (e.g., database, storage array, network storage device, etc.) accessible by the computer 62 via an external connection. The processor 64 may be configured to control any of the methods and/or processes described herein and/or to cause such methods, and/or processes to be performed, e.g., by the cryoablation console 14. Processor 64 corresponds to one or more processors for functions described herein. Memory is configured to store data, programmatic software code and/or other information described herein. In some embodiments, the software may include instructions that, when executed by the processor 64 causes the processor 64 to perform the processes described herein with respect to the cryoablation system 10.

The user input devices 66 may include keyboards, knobs, buttons, dials, foot pedals, mice, touchscreens, voice input units, and/or switches. Additionally, the user may use the user input devices 66 to override the automatic operation of the system 10 either programmed into or predetermined by the cryoablation console 14. Still further, signals received by the one or more processors 64 may be used to automatically or semi-automatically control the configuration of the balloon 48 (for example, by extending or retracting the actuation element 52). The one or more computers 62 may further include one or more displays 68, such as computer screens or other visual elements in communication with the one or more processors 64 and/or user input devices 66. Finally, the cryoablation console 14 may include one or more speakers or other audio alert generators that are in communication with the one or more processors 64 and/or the user input devices 66.

The catheter 12 may include a handle 70 coupled to the proximal portion 42 of the elongate body 41. The handle 70 can include circuitry for identification and/or use in controlling of the catheter 12 or another component of the system 10. For example, the handle 70 may include one or more sensors to monitor system parameters such as the refrigerant pressure within the catheter 12 or one or more refrigerant flow paths of the system. In some embodiments, the handle 70 may include the thermocouple 40A and/or pressure transducer 40B. Additionally, the handle 70 may also include connectors 72 that can be directly mated to a refrigerant supply conduit from the injection system 20 via a supply port of the cryoablation console 14 and or the cryoablation cooling system 18. The connectors 72 of the handle 70 may also be directly mated to an exhaust port on cryoablation console 14. Alternatively, the connectors 72 may be coupled to the injection system 20 and/or the exhaust port on the cryoablation console 14 or an exhaust conduit indirectly by way of one or more umbilical (flexible) conduits. The handle 70 may include a pressure relief valve in fluid communication with the refrigerant delivery conduit 44 and/or exhaust lumen to automatically open when a predetermined pressure threshold value is exceeded.

The handle 70 may also include one or more actuation or control features that allow a user to control, deflect, steer, or otherwise manipulate the distal portion 15 of the medical device from the proximal portion 42 of the catheter 12. For example, the handle 70 may include one or more components such as a lever or knob 74 for manipulating the elongate body 41, actuation element 52, a guidewire, and/or additional components of the catheter 12.

The catheter 12 may further include one or more sensors 76 to monitor operating parameters, including thermocouple 40A and/or pressure transducer 40B, for example. Also, note that one thermocouple 40A and/or pressure transducer 40B to measure refrigerant temperature and/or refrigerant pressure may be placed in the catheter 12 and another thermocouple 40A and/or pressure transducer 40B to measure refrigerant temperature and/or refrigerant pressure may be placed at the heat exchanger 38. Placing the sensors 40A and/or 40B within the catheter 12 may provide more accurate determination of the pressure and temperature of the refrigerant that is delivered to the point of treatment.

The sensors 76 may include a pressure sensor and a temperature sensor (for example, thermocouple 40A/pressure transducer 40B) to detect a pressure and temperature, respectively, of the refrigerant entering the catheter 12 from the injection system 20 and to produce signals representative of sensed pressure and temperature. These signals may be routed to the sub-cooler control system 26 as feedback to determine a pressure and/or temperature set point that the sub-cooler controller 32 will seek to maintain.

The sensor(s) 76 may be in communication with the cryoablation console 14 for initiating or triggering one or more alerts or therapeutic delivery modifications during operation of the medical device 12. One or more valves, controllers, or the like may be in communication with the sensor(s) 76 to provide for the controlled dispersion or circulation of refrigerant through the lumens/fluid paths of the catheter 12 and system 10. Such valves, controllers, or the like may be located in a portion of the catheter 12 and/or in the cryoablation console 14.

While the catheter 12 may be in fluid communication with a refrigerant source 22 to cryogenically treat selected tissue, it is also contemplated that the catheter 12 may additionally include one or more electrically conductive portions or electrodes thereon coupled to a radiofrequency generator or electrical power source 78 as a treatment or diagnostic mechanism. If the catheter 12 includes thermoelectric cooling elements or electrodes capable of transmitting radiofrequency (RF), ultrasound, microwave, electroporation energy, or the like, the elongate body 41 may include a lumen in electrical communication with the power source 78.

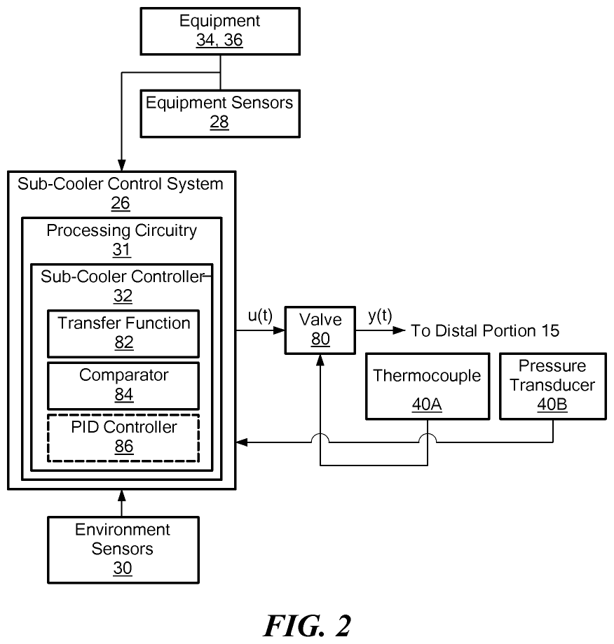

FIG. 2 is a block diagram of an example control loop that includes the sub-cooler control system 26. The sub-cooler control system 26 includes the sub-cooler processing circuitry 31 which is configured to implement the sub-cooler controller 32 to monitor operating parameters and environment parameters sensed by the equipment sensors 28, thermocouple 40A, pressure transducer 40B and the environment sensors 30, and to regulate an operating parameter such as the temperature and/or pressure of the refrigerant received by the catheter 12. The refrigerant temperature at the output of the heat exchanger 38 may be regulated by controlling a valve 80 that controls the flow of coolant to the heat exchanger 38. Alternatively, or in addition, the refrigerant pressure may be controlled by a valve 80 or other device that controls the pressure or flow rate of the refrigerant, in which case valve 80 may be or include a pressure regulator that may be located at the injection system 20, for example.

In operation, the sub-cooler controller 32 receives signals representative of operating parameters of the equipment 34 and 36, and signals representative of environmental operating parameters from sensors 28 and 30, respectively. The sub-cooler controller 32 includes a transfer function unit 82, a comparator 84 and optionally, a sub-cooler PID controller 86. The transfer function unit 82 implements a transfer function that relates measured and calculated operational parameters to an output, y(t). The output y(t) may be a signal representative of a predicted temperature or pressure of the refrigerant or a cooling power of the refrigerant. An example of a such a transfer function is as follows:

$$y ( t ) = I + {k}_{1} A + {k}_{2} B + {k}_{3} C + {k}_{4} D + k 5 AB + {k}_{6} A C + {k}_{7} A D + {k}_{8} BC + {k}_{9} BD + {k}_{10} CD + {k}_{11} {A}^{2} + {k}_{12} {B}^{2} + {k}_{13} {C}^{2} + {k}_{14} {D}^{2}$$

where: A is the coolant charge pressure, B is the environment temperature, C is the speed of the compressor 34 and D is the condenser fan speed. The coefficients, k, may be determined based on prior experimentation. For example, a number of values of a measured refrigerant parameter, y(t), (such as pressure, temperature or cooling power) may be determined by measurement of the refrigerant parameter for each of a number of sets of values of A, B, C and D. From the point of view of numerical computation, the multiple sets of values of A, B, C and D and resultant values of the refrigerant parameter may be considered conceptually as defining points on a surface in a multidimensional space, with the coefficients k being determined by curve-fitting, for example, so that the transfer function y(t) approximates a contour of the surface. Perturbation methods may also be employed to determine the transfer function y(t).

Thus, the transfer function may be approximated by a linear or quadratic function, for example, of the operating parameters and environment parameters that produces a refrigerant parameter such as refrigerant temperature, refrigerant pressure or cooling power of the refrigerant as an output. Higher order transfer functions may be implemented in some embodiments, at the possible extent of greater computational complexity.

Once the transfer function y(t) is determined, the transfer function may be used to control the refrigerant parameter (pressure, temperature, cooling power). For example, the parameters C and D (compressor speed and condenser fan speed), may be determined from the transfer function that produces a target refrigerant parameter, yₜ(t), for a given coolant charge pressure A and environment temperature B. The compressor speed and condenser fan speed may then be set to the determined values C and D, respectively. This results in an actual refrigerant parameter yₘ(t) that is measured by the thermocouple 40A and/or transducer 40B. A comparator 84 may then compare the target refrigerant parameter yₜ(t) to the measured refrigerant parameter yₘ(t) to produce an error signal, e(t)=yₘ(t)−yₜ(t). New values of C and D can then be determined that drives the error signal e(t) toward zero.

In some embodiments, the sub-cooler controller 32 may include a sub-cooler PID controller 86 which is configured to determine an output u(t) which depends on the error signal e(t) and which drives the valve 80 to control the flow rate of the refrigerant to the distal portion 15 of the cryoablation catheter. The sub-cooler PID controller 86 is a control circuit that employs a sum of a constant Kp times an error signal, a constant Ki times an integral of the error signal, and a constant Kd times a derivative of the error signal. These constants, Kp, Ki, and Kd, as well as the integration interval over the error signal, may be determined by the sub-cooler processing circuitry 31 based at least in part on the operational parameters received by the equipment sensors 28, the environment sensors 30 and the thermocouple 40A/pressure transducer 40B.

Thus, inputs to the sub-cooler control system 26 may include, for example, a flow rate set point chosen to achieve an operator-selected refrigerant temperature, an input pressure of the refrigerant source 22, an output pressure of the injection system 20 measured by, for example the pressure transducer 40B, a refrigerant injection temperature measured by, for example the thermocouple 40A, and a console temperature inside the console measured by an equipment sensor 28 or an environment sensor 30. Other inputs to the sub-cooler control system 26 may include, for example, a time duration of integration, a flow rate of the coolant delivered to the heat exchanger 38, a coolant charging pressure, a condenser fan speed and a compressor speed. Advantages to including more sensor inputs to the sub-cooler control system 26 may include increased stability of the refrigerant parameter in responses to changes in the sensed parameters.

Outputs of the sub-cooler controller 32 may include messages that are sent to the computer 68 to be displayed on the display 62. These messages may include error messages such as an indication of catheter temperature overshoot or undershoot, an indication that the refrigerant flow rate is too high or too low, an indication of an unallowable or unachievable target refrigerant power set by the operator, an indication that an operating temperature of the cryoablation console 14 is too high or too low, and an indication of off and on states of the injection system 20.

The processing circuitry 31, including the sub-cooler controller 32 may be implemented as a computer processor executing computer instructions stored in memory and/or by application specific circuitry, such as a central processing unit, and memory and/or integrated circuitry for processing and/or control, e.g., one or more micro-processors and/or processor cores and/or FPGAs (Field Programmable Gate Array) and/or ASICs (Application Specific Integrated Circuitry) adapted to execute instructions. The sub-cooler processing circuitry 31 may be configured to access (e.g., write to and/or read from) the memory, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

Software executed by the sub-cooler controller 32 may be stored in a memory located in proximity to the sub-cooler controller 32 or stored in external memory (e.g., database, storage array, network storage device, etc.) accessible by the sub-cooler controller 32 via an external connection. The sub-cooler controller 32 may be configured to control any of the methods and/or processes attributable to the sub-cooler controller 32 and/or to cause such methods, and/or processes to be performed. The sub-cooler controller 32 may correspond to one or more processors for performing functions described herein. Memory is configured to store data, programmatic software code and/or other information described herein. In some embodiments, the software may include instructions that, when executed by the sub-cooler controller 32 causes the sub-cooler controller 32 to perform the processes described herein with respect to operation of the sub-cooler control system 26.

Thus, according to one aspect, a cryoablation cooling system 18 includes: cooling equipment 34, 36, having at least one operating parameter; at least one external environment sensor, each of the at least one external environment sensors 30 being configured to measure an environmental parameter external to the cooling equipment 34, 36; at least one equipment sensor 28, each of the at least one equipment sensor 28 being configure to measure an operating parameter of the cooling equipment 34, 36; and a sub-cooler control system 26 configured to determine an adjustment to at least one operating parameter of the cooling equipment 34, 36 to obtain a target refrigerant parameter based at least in part on the at least one measured external environmental parameter and the at least one measured operating parameter.

According to this aspect, in some embodiments, the adjustment is based on a transfer function that relates a given set of operating parameters and external environment parameters to a target refrigerant parameter. In some embodiments, the transfer function is predetermined based on measurements of the target refrigerant parameter for a number of sets of operating parameters. In some embodiments, determining at least one operating parameter based on the transfer function is further based on a difference between the target refrigerant parameter and a measured refrigerant parameter. In some embodiments, determining the adjustment includes: setting the target refrigerant parameter; and determining at least one operating parameter setting based on the transfer function and the set target refrigerant parameter. In some embodiments, the at least one measured operating parameter is one of a condenser fan speed, a compressor speed and a coolant charging pressure. In some embodiments, the sub-cooler control system includes a sub-cooler proportional-integral-derivative (PID) controller configured to determine a control signal based at least in part on the target refrigerant parameter and a measured refrigerant parameter, the control signal controlling an operational parameter of the cooling equipment 34, 36. In some embodiments, the target cooling power is determined based at least in part on a transfer function having inputs that include at least one measured operating parameter and at least one measured external environmental parameter. In some embodiments, the refrigerant parameter is one of a refrigerant pressure, a refrigerant temperature, and a refrigerant cooling power. In some embodiments, the at least one measured operating parameter is a pressure measured by a transducer and the at least one measured external environmental parameter is a temperature measured by a thermocouple.

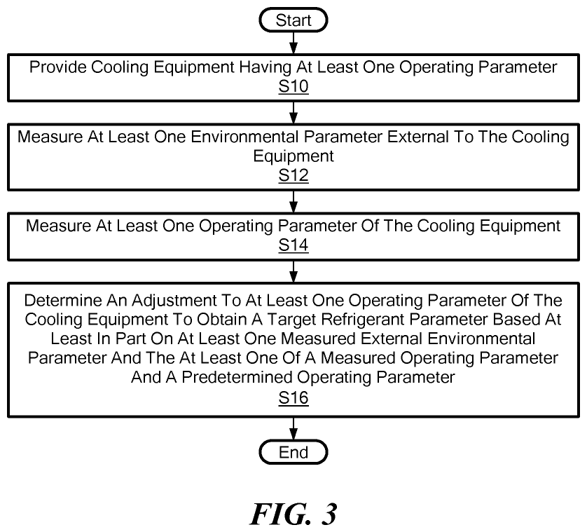

FIG. 3 is a flowchart of an example process in a cryoablation cooling system 18 that may be performed at least in part by the sub-cooler control system 26, equipment sensors 28, environment sensors 30 and thermocouple 40A/pressure transducer 40B. The process includes providing cooling equipment 34, 36 having at least one operating parameter (Block S10). The process also includes measuring at least one environmental parameter external to the cooling equipment 34, 36 (Block S12), and measuring at least one operating parameter of the cooling equipment 34, 36 (Block S14). The process further includes determining an adjustment to at least one operating parameter of the cooling equipment 34, 36 to obtain a target refrigerant parameter based at least in part on at least one measured external environmental parameter and at least one of a measured operating parameter and a predetermined operating parameter (Block S16).

In some embodiments, the adjustment is based on a transfer function that relates a given set of operating parameters and external environment parameters to a target refrigerant parameter. In some embodiments, the transfer function is predetermined based on measurements of the target refrigerant parameter for a number of sets of operating parameters. In some embodiments, determining at least one operating parameter based on the transfer function is further based on a difference between the target refrigerant parameter and a measured refrigerant parameter. In some embodiments, determining the adjustment includes: setting the target refrigerant parameter; and determining at least one operating parameter setting based on the transfer function and the set target refrigerant parameter. In some embodiments, the at least one measured operating parameter is one of a condenser fan speed, a compressor speed and a charging pressure. In some embodiments, the sub-cooler control system 26 includes a sub-cooler proportional-integral-derivative (PID) controller configured to determine a control signal based at least in part on the target refrigerant parameter and a measured refrigerant parameter, the control signal controlling an operational parameter of the cooling equipment 34, 36. In some embodiments, the target cooling power is determined based at least in part on a transfer function having inputs that include at least one measured operating parameter and at least one measured external environmental parameter. In some embodiments, the refrigerant parameter is one of a refrigerant pressure, a refrigerant temperature, and a refrigerant cooling power. In some embodiments, the at least one measured operating parameter is a pressure measured by a transducer and the at least one measured external environmental parameter is a temperature measured by a thermocouple.

In some embodiments, a process for correcting for environmental changes includes:
Input a known compressor charge setpoint to the transfer function;
Read environment parameters (for example, room temperature);
Determine operating parameters by solving the transfer function for the operating parameters; and
Adjust equipment operating parameters according to the determined operating parameters to achieve and maintain a desired refrigerant parameter (such as temperature and pressure).

It has been tested and shown that the environmental temperature surrounding the cryoablation console 14 impacts cooling power. This impact changes for different flow rates and console types across the range of the operating temperatures of the cryoablation console 14. For the system to deliver true robust therapy and consistent cooling power, the targeted flow rate and the parameters of the sub-cooler changes with the surrounding environment temperature. In some embodiments, a known charge pressure of the coolant and measured operating parameters and environment parameters are used to control a coolant parameter to maintain the refrigerant parameter within a narrow range of a mean value.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope of the invention, which is limited only by the following claims.

What is claimed is:

1. A cryoablation cooling system, comprising:
cooling equipment having at least one operating parameter;
at least one external environment sensor, each of the at least one external environment sensors being configured to measure an environmental parameter external to the cooling equipment;
at least one equipment sensor, each of the at least one equipment sensor being configure to measure an operating parameter of the cooling equipment; and
a sub-cooler control system configured to determine an adjustment to at least one operating parameter of the cooling equipment to obtain a target refrigerant parameter based at least in part on the at least one measured external environmental parameter and at least one of a measured operating parameter and a predetermined operating parameter.

2. The system of claim 1, wherein the adjustment is based on a transfer function that relates a given set of operating parameters and external environment parameters to a target refrigerant parameter.

3. The system of claim 2, wherein the transfer function is predetermined based on measurements of the target refrigerant parameter for a number of sets of operating parameters.

4. The system of claim 3, wherein determining at least one operating parameter based on the transfer function is further based on a difference between the target refrigerant parameter and a measured refrigerant parameter.

5. The system of claim 2, wherein determining the adjustment includes:
setting the target refrigerant parameter; and
determining at least one operating parameter setting based on the transfer function and the set target refrigerant parameter.

6. The system of claim 1 wherein the at least one measured operating parameter is one of a condenser fan speed, a compressor speed and a charging pressure.

7. The system of claim 1, wherein the sub-cooler control system includes a sub-cooler proportional-integral-derivative (PID) controller configured to determine a control signal based at least in part on the target refrigerant parameter and a measured refrigerant parameter, the control signal controlling an operational parameter of the cooling equipment.

8. The system of claim 6, wherein the target cooling power is determined based at least in part on a transfer function having inputs that include at least one measured operating parameter and at least one measured external environmental parameter.

9. The system of claim 1, wherein the refrigerant parameter is one of a refrigerant pressure, a refrigerant temperature, and a refrigerant cooling power.

10. The system of claim 1, wherein the at least one measured operating parameter is a pressure measured by a transducer and the at least one measured external environmental parameter is a temperature measured by a thermocouple.

11. A method in a cryoablation cooling system, the method comprising:
providing cooling equipment having at least one operating parameter;
measuring via at least one external environment sensor an environmental parameter external to the cooling equipment;
measuring via at least on equipment sensor an operating parameter of the cooling equipment; and
determining an adjustment to at least one operating parameter of the cooling equipment to obtain a target refrigerant parameter based at least in part on the at least one measured external environmental parameter and at least one of a measured operating parameter and a predetermined operating parameter.

12. The method of claim 11, wherein the adjustment is based on a transfer function that relates a given set of operating parameters and external environment parameters to a target refrigerant parameter.

13. The method of claim 12, wherein the transfer function is predetermined based on measurements of the target refrigerant parameter for a number of sets of operating parameters.

14. The method of claim 13, wherein determining at least one operating parameter based on the transfer function is further based on a difference between the target refrigerant parameter and a measured refrigerant parameter.

15. The method of claim 12, wherein determining the adjustment includes:
setting the target refrigerant parameter; and
determining at least one operating parameter setting based on the transfer function and the set target refrigerant parameter.

16. The method of claim 11 wherein the at least one measured operating parameter is one of a condenser fan speed, a compressor speed and a charging pressure.

17. The method of claim 11, wherein the sub-cooler control system includes a sub-cooler proportional-integral-derivative (PID) controller configured to determine a control signal based at least in part on the target refrigerant parameter and a measured refrigerant parameter, the control signal controlling an operational parameter of the cooling equipment.

18. The method of claim 16, wherein the target cooling power is determined based at least in part on a transfer function having inputs that include at least one measured operating parameter and at least one measured external environmental parameter.

19. The method of claim 11, wherein the refrigerant parameter is one of a refrigerant pressure, a refrigerant temperature, and a refrigerant cooling power.

20. The method of claim 11, wherein the at least one measured operating parameter is a pressure measured by a transducer and the at least one measured external environmental parameter is a temperature measured by a thermocouple.
